(19) United States
(12) Patent Application Publication
(10) Pub. No.: US 2024/0164655 A1
(43) Pub. Date: May 23, 2024

(54) Neurological Assessment Device

(71) Applicant: Zaid Aljuboori, Wilkes Barre, PA (US)

(72) Inventor: Zaid Aljuboori, Wilkes Barre, PA (US)

(21) Appl. No.: 18/237,466

(22) Filed: Aug. 24, 2023

Related U.S. Application Data
(60) Provisional application No. 63/384,414, filed on Nov. 19, 2022.

(51) Int. Cl. A61B 5/0531; A61B 5/00

(52) U.S. Cl. CPC A61B 5/0531; A61B 5/4047; A61B 5/4824

(57) ABSTRACT

An electrical stimulation device provides a metered electrical stimulation to the epidermis largely eliminating inter-examiner variability to provide improved longitudinal measurements with reduced patient risk during neurological examinations.

### CROSS REFERENCE TO RELATED APPLICATION

This application claims the benefit of U.S. provisional application 63/384,414 filed Nov. 19, 2022, and hereby incorporated by reference.

### STATEMENT REGARDING FEDERALLY SPONSORED RESEARCH OR DEVELOPMENT

### BACKGROUND OF THE INVENTION

The present invention relates generally to devices for use in neurological examination of patients with brain injuries or the like and, in particular, to a device providing a repeatable, standardized stimulus for such assessment.

Frequent neurological examination of patients with brain injury and altered neurological status is important in providing comprehensive care. The Glasgow Coma Scale (GCS) is a method that was developed to assess the conscious state of such patients. Three types of responses to a stimulus are measured: best motor response (1 to 6), best verbal response (1 to 5), and eye opening (1 to 4). The sum of these scores separate patients into normal, mild, moderate, and severe brain injury categories with low scores indicating poor conscious status.

Currently, a variety of types of stimulus are employed including sternal rubbing, skin pinching, or nail bed pressure. The healthcare professional observes and quantifies a patient's response to a stimulus and determines the patient's neurological status.

While current methods of stimulation are effective, they have drawbacks. Each of these stimulus methods is subject to inter-examiner variability such as may produce inconsistent results. Improperly applied, such stimuli may physically damage the tissues increasing the risk for skin infection. While the stimulus is necessary diagnostically, it also can lead to emotional distress in observers such as the patient's family.

### SUMMARY OF THE INVENTION

The present invention provides a safe electrical stimulation for consistent neurological measurements important in making longitudinal assessments. The system provides a depth-limited stimulation to the free nerve endings (FNE) of the epidermis being unspecialized, un-encapsulated sensory afferent nerve endings used by vertebrates to detect pain. By providing a shallow stimulation with precisely metered electrical energy, a repeatable neurological evaluation can be obtained.

In one embodiment, the neurological stimulation device provides an intra-epidermal skin electrode array adapted to be placed against the skin and an electrical switch providing an activation signal. An electrical energy source communicates with the electrode array and the electrical switch and responds to the activation signal to provide a metered discharge of electricity to the electrode array to produce an intra-epidermal electrical stimulation concentrated in the epidermis with a duration of less than one second and a current of greater than 4 mA.

It is thus a feature of at least one embodiment of the invention to provide an alternative to traditional neurological stimulation techniques, providing more consistent evaluation without risk of patient injury or an appearance of patient mistreatment.

The neurological stimulation device may further include a monitoring circuit monitoring the electrodermal activity of the skin to provide an output indicating that activity in response to the metered discharge of electricity.

It is thus a feature of at least one embodiment of the invention to provide a quantitative measurement of patient response to stimulation.

The intra-dermal skin electrode array may provide a set of needle electrodes extending from a stop surface to penetrate the epidermis to a depth stopping above the dermis.

It is thus a feature of at least one embodiment of the invention to confine the stimulation to the epidermis to promote stimulation without tissue injury.

The needle electrodes may extend less than 1.5 mm from the stop surface.

It is thus a feature of at least one embodiment of the invention to allow robust limitation of the depth of placement of the electrodes by limiting their length to a typical epidermal thickness.

The electrode array may provide a releasable electrical connector communicating between the electrode array and a housing holding the electrical switch and the electrical energy source.

It is thus a feature of at least one embodiment of the invention to provide a simple handheld unit that can be used to provide the desired electrical stimulation.

The electrical energy source maybe a current source providing a variable voltage to produce a constant current through the epidermis;

It is thus a feature of at least one embodiment of the invention to provide for consistent electrical stimulation with different skin characteristics and electrode coupling.

The neurological stimulation device may further include a timing circuit controlling a time duration of the meter discharge of electricity for a predetermined duration after the activation signal and locking out the meter discharge of electricity for a second predetermined time after the meter discharge of electricity.

It is thus a feature of at least one embodiment of the invention to provide a lockout feature preventing accidental multiple stimulation prior to assessing response.

These particular objects and advantages may apply to only some embodiments falling within the claims and thus do not define the scope of the invention.

### BRIEF DESCRIPTION OF THE DRAWINGS

FIG. 1 is a perspective view of a portable neurological stimulation device according to one embodiment of the invention having a handheld housing releasably attached to a stimulating electrode array and providing a connector for a secondary skin response array;

FIG. 2 is an elevational cross-section through the stimulating electrode array showing constrained depth needle electrodes to promote epidermal current flow removed from the dermis;

FIG. 3 is a bottom plan view of the stimulating electrode array of FIG. 2 showing a central return electrode surrounded by stimulating electrodes;

FIG. 4 is an elevational cross-sectional view similar to that of FIG. 2 showing the secondary skin response array;

FIG. 5 is a bottom plan view of the skin response array of FIG. 4 providing topical electrodes;

FIG. 6 is a schematic diagram of the circuitry of the portable neurological stimulation device providing an internal microcontroller and battery; and

FIG. 7 is a timing diagram showing the discharge of electrical energy into the stimulating electrode array as may be provided by the present invention in one embodiment.

### DETAILED DESCRIPTION OF THE PREFERRED EMBODIMENT

Referring now to FIG. 1, a neurological stimulator 10 according to the present invention may provide a handheld housing 12 providing a display 14, for example, a backlit liquid crystal or LED display, and various user controls including a stimulation button 16 and a skin activity button 18 as will be operationally described in more detail below. Additional user controls 20 on the housing 12 allow the user to preset an amount of the electrical stimulation produced by the neurological stimulator 10. An on-off switch 22 provides connection to an internal battery, and a set of front connectors 26 are provided for additional electrical connections to electrode arrays as will be discussed.

In one embodiment, the bottom of the housing 12 may provide an electrical and mechanical connector 28 providing separable halves that, when joined, allow communication between circuitry within the housing 12 and a stimulating electrode array 30. The stimulating electrode array 30, as so attached, may be positioned against the skin 32 of a patient for stimulation thereof.

A separate skin response electrode 34 may also be placed on the skin 32 and may communicate with the circuitry within the housing 12 via one of a set of frontwardly exposed electrical connectors 26. Various other of the electrical connectors 26 allow for the connection of additional stimulating electrode arrays 30 or skin response electrodes 34 as may be desired.

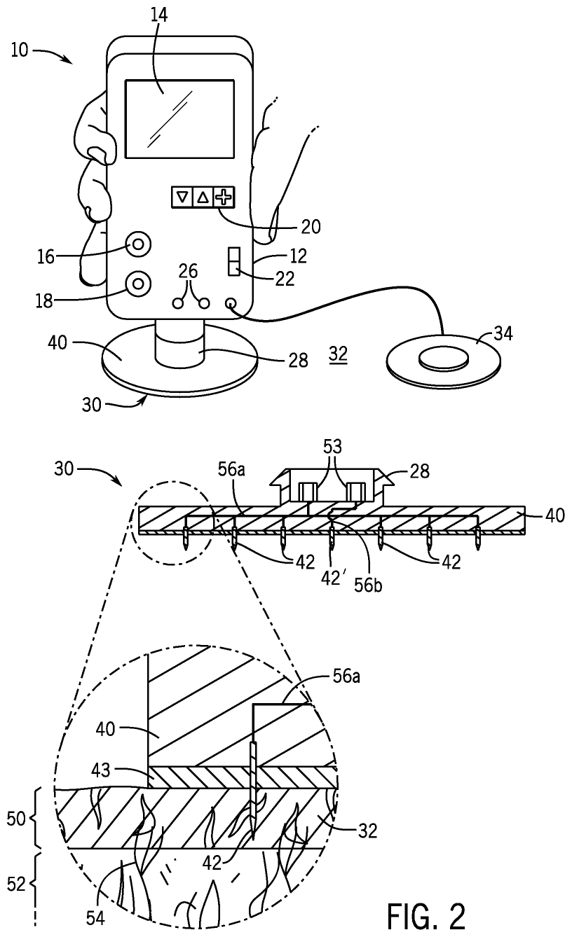

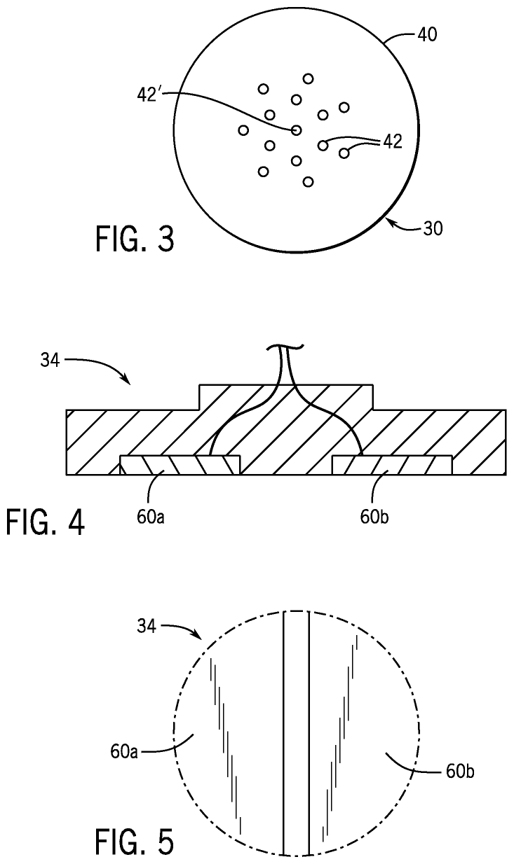

Referring now to FIGS. 2 and 3, the stimulating electrode array 30 may provide, for example, a rigid insulating disk 40, for example, of an electrically insulating polymer material. A set of needle electrodes 42 are held by the disk 40 positioned to extend downwardly from the disk 40 toward the skin 3 within a diameter of 1 cm or the like. In one embodiment, the stimulating electrode array 30 employs a central needle electrode 42′ providing an electrical return (cathode) for stimulating current flowing from a set of surrounding electrodes 42. Electrical conductors 55a and 55b serve to connect respective groups of the electrodes 42 and the individual electrode 42′ to respective connections 53 in and attached to half of the mechanical connectors 28.

The invention contemplates that the insulating disk 40 may provide 5 to 15 stainless steel electrodes 42, spaced from 2 to 10 mm apart, measuring 0.5 mm in diameter with a 0.2 mm length of extension. The electrodes 42 and 42′ may extend from the insulating disk 40 through a soft cushion layer 43 such as an elastomer into the epidermis 50 terminating before the dermis 52. In this way electrodes 42 primarily stimulate the free nerve endings 54 located in the epidermis 50, while avoiding mechanoreceptors that are mainly located deeper in the dermis 52. As a result, the pulses of electric current between the electrodes 42 and the central electrode 42′ are volumetrically restricted to the epidermis 50 to selectively activate the fast pain nociceptors.

In one embodiment, the electrodes 42 may be electrically stimulated in a session defined by two or more pulses having a current intensity from 0.1-10 mA and importantly including values above 2 mA and above 4 mA such as may be necessary for patients with altered mental status. The current may be either biphasic or monophasic and may provide for a setting selecting between these two modes. During the pulse the frequency may be greater than 30 Hz in the interval between pulses may be from 1 to 400 ms. Generally, the pulse stimulations will last from 0.5 to 300 ms.

Referring now to FIGS. 4 and 5, the skin response electrode 34 may provide for a pair of planar electrode surfaces 60a and 60b, for example, constructed in the manner of EKG electrodes or the like for topical application to measure skin response such as galvanic skin response (GSR). The skin response electrodes 34 may provide a measure of a patient's reaction to the stimuli from the stimulating electrode array 30 in otherwise nonresponsive patients.

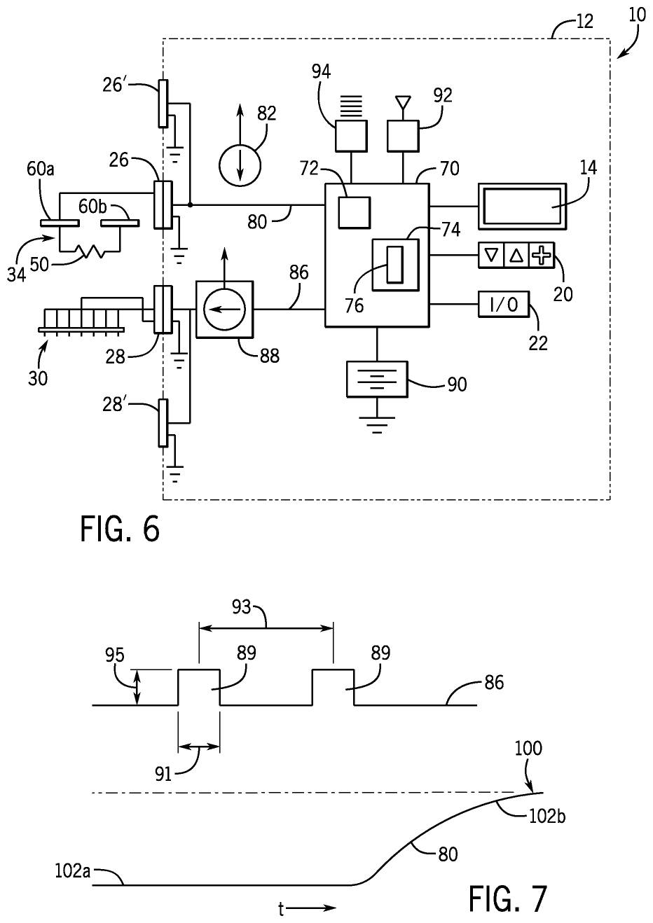

Referring now to FIG. 6, control of the operation of the neurological stimulator 10 may be implemented through a microcontroller 70, for example, including one or more internal processors 72 communicating with electronic memory 74 holding a stored program 76. The microcontroller 70 may provide for an internal analog-to-digital converter for receiving a galvanic signal 80 from the skin response electrode 34, for example, measuring a resistive voltage drop across the skin between the electrode surfaces 60a and 60b when subjected to a steady-state current from a current source 82. As is understood in the art, a current source is a circuit that monitors current and adjusts its voltage to provide a desired current level.

The microcontroller 70 executing the stored program 76 may also produce a digital output control signal 86 controlling a current source 88 providing pulses of electrical current to the stimulating electrode array 30. In one embodiment, two or more pulses 89 may be selected for delivery to the patient according to settings previously entered through user controls 20.

Referring also to FIG. 7, these pulses 89 may have a current intensity 95 of 0.01-4 mA and a duration 91 of from 0.5 to 100 ms and may have a spacing 93 between 5 and 25 ms. Generally, the pulse 89 will be triggered by a user pressing the stimulation button 16 and will be locked out for a period of time after each stimulation from 5 to 30 seconds to prevent accidental re-triggering. After stimulation, a pressing of the skin activity button 18 may cause the microcontroller 70 to read the galvanic skin response 100 (here showing conductance) to provide a quantitative output on the display 14 comparing values 102a before and 102b after stimulation.

The housing 12 may support a rechargeable battery 90 providing power to the display 14, the controller 70, the current source 82, and the current source 88. Generally, the current source 88 may include a DC-DC converter for boosting the battery voltage to an amount sufficient to obtain the desired current flow through the skin resistance.

In some embodiments, the housing 12 may also support a wireless transceiver 92, for example, allowing the neurological stimulator 10 to communicate with remote computers or handheld devices such as a cell phone or the like for input or output of data including, for example, recording patient identifiers, date, time, stimulation settings, skin response measurements, and Glasgow Coma Scale diagnoses. A barcode reader 94 may also be provided to quickly set the various parameters of current stimulation discussed above without the need for entry through user controls 20 such as a keypad or touchscreen.

It will be appreciated that the function of the stimulating electrode array 30 and skin response electrode 34 may alternatively be combined and that additional electrodes of each of these types may be connected by the connectors 26 discussed above.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microcontroller” and “a processor” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What I claim is:

1. A neurological stimulation device comprising:
an intra-epidermal skin electrode array adapted to be placed against the skin of a patient;
an electrical switch providing an activation signal; and
an electrical energy source communicating with the electrode array and the electrical switch and responding to the activation signal to provide a metered discharge of electricity to the electrode array to produce an intra-epidermal electrical stimulation concentrated in the epidermis of the skin with a duration of less than one second at greater than 4 mA.

2. The neurological stimulation device of claim 1 further including a monitoring circuit monitoring electrodermal activity of the skin to provide an output indicating that activity in response to the metered discharge of electricity.

3. The neurological stimulation device of claim 1 wherein the intra-epidermal skin electrode array provides a set of needle electrodes extending from a stop surface to penetrate the epidermis to a depth stopping above the dermis.

4. The neurological stimulation device of claim 3 wherein the needle electrodes are less than 1.5 mm in length from the top surface.

5. The neurological stimulation device of claim 3 wherein the needle electrodes include electrodes receiving the metered discharge of electricity and electrodes providing an electrical return to the electrical energy source.

6. The neurological stimulation device of claim 3 wherein the monitoring circuit monitors electrodermal activity using a monitoring electrode array different from the intra-epidermal skin electrode array.

7. The neurological stimulation device of claim 1 wherein the electrode array includes a releasable electrical connector communicating between the electrical array and a housing holding the electrical switch and the electrical energy source.

8. The neurological stimulation device of claim 1 wherein the electrical energy source is a current source providing a variable voltage to produce a constant current through the epidermis;

9. The neurological stimulation device of claim 1 further including a timing circuit controlling a time duration of a metered discharge of electricity for a predetermined duration after the activation signal and locking out the meter discharge of electricity for a second predetermined time after the meter discharge of electricity.

10. The neurological stimulation device of claim 1 wherein the intra-epidermal skin electrode array, electrical switch and electrical energy source are integrated with a single handheld housing providing a rechargeable battery providing energy to the electrical energy source.

11. The neurological stimulation device of claim 1 wherein including a user interface allowing a setting of the electrical energy source to provide a current intensity at multiple levels.

12. The neurological stimulation device of claim 1 wherein including a user interface allowing a setting of the electrical energy source to provide a meter discharge of electricity for a duration of 0.5 to 100 ms.
